(19) United States
(12) Patent Application Publication
(10) Pub. No.: US 2006/0078139 A1
(43) Pub. Date: Apr. 13, 2006

(54) Method for adapting a hearing device to a momentary acoustic surround situation and a hearing device system

(76) Inventors: Hilmar Meier, Zurich (CH); Michael Boretzki, Ruti (CH)

Correspondence Address: PEARNE & GORDON LLP, 1801 EAST 9TH STREET, SUITE 1200, CLEVELAND, OH, 44114-3108, US

(21) Appl. No.: 10/400,842

(22) Filed: Mar. 27, 2003

(51) Int. Cl. H04R 29/00; H04R 25/00; A61B 5/00

(52) U.S. Cl. 381/312; 381/60; 600/559

(57) ABSTRACT

In a signal processing unit (a) of a hearing device (1) the signal processing unit (8) consisting of a signal analyzing unit (9) and a signal identification unit (10), the momentary acoustic surround situation is identified. Based on this identified surroun situation, the parameter set (7) belonging to the identified surround situation is automatically adjusted in a transmission unit (5) in which a number of parameter sets are saved which are assigned to different acoustic surround situation. The hearing device user can adjust his individual hearing desire for each acoustic surround situation according to the automatic adjusted parameter set with the aid of an input unit (11). Thereby, the parameter of this parameter set are adjusted at the same time and commnnly in one of two opposing directions, e.g. in the sense of “better hearing” or in the sense of “more pleasant understanding”.

The present invention is related to a method for adapting a hearing device to a momentary acoustic surround situation according to the pre-characterizing part of claim 1, as well as to a hearing device system according to the pre-characterizing part of claim 8.

A method and a hearing device system of this kind are known from WO-A-01/20965 or from the corresponding US-A-2002/0037087. By this known solution, the momentary acoustic surround situation is identified in the hearing device of the hearing device system, and the corresponding hearing program, i.e. the corresponding set of parameters saved in the hearing device, is automatically adjusted according to the identified acoustic surround situation. In order to provide the possibility to the hearing device user to switch off, as he wishes, the automatic recognition of the acoustic surround situation and the corresponding automatic selection of the hearing program, an input unit, e.g. a switch at the hearing device or at a remote control, is provided which can be operated by the hearing device user.

Besides that, hearing devices are also known for which the selection of the suitable hearing program can be manually made by the hearing device user from a number of hearing programs saved in the hearing device, and, to be precise, over a remote control or over a switch at the hearing device. For a lot of users, the switching between different hearing programs is however tiresome or difficult, in particular then when a lot of hearing programs are provided for selection,

The present invention has therefore the object to Provide a method or a hearing device system, respectively, of the kind mentioned above, for which the hearing program corresponding to the moments acoustic surround situation is automatically adjusted but that the hearing device user has the possibility to introduce his personal hearing wish without great expenditure in certain Bituations.

This object is achieved according to the present invention by a method having the elements of claim 1 and by a hearing device system with the elements of claim 8.

The hearing device user can therefore take influence on the hearing program automatically selected by the hearing device of the hearing device systemn, i.e. by the set of paramneters corresponding to the selected hearing program, in that the hearing device user adjusts the parameters with the aid of an input unit sliply operable according to predefined rules saved in the hearing device. Therewith, the hearing device user is capable to adjust the hearing program in every surround situation in an easy marirler, the hearing prcarn being adjusted to this specific surround situation automatically and according to his personal desire, as e.g. in the sense of “better understanding” or “more-pleasant hearing”.

Preferably, the parameters of the adjusted parameter set are adjusted starting from an adjusted nominal value within a preset rule range according to preset rules in one of two opposing directions.

Preferred embodiments of the method according to the present invention and of the hearing device system according to the present invention are subject to dependent claims.

In the following, the invention is further described along with figures. Therein, it is schematically shown:

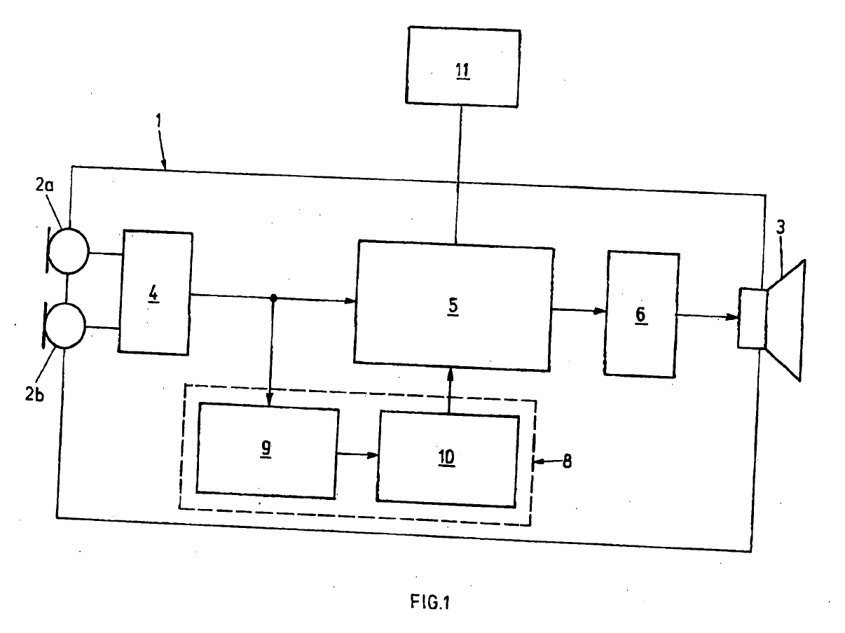

FIG. 1 a block diagram of a hearing device system according to the present invention,

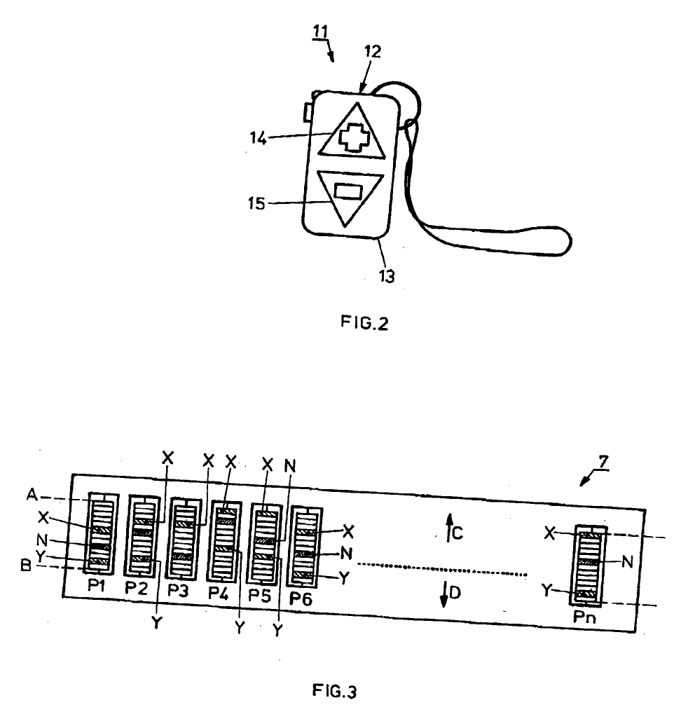

FIG. 2 an embodiment of an input unit belonging to the hearing device system according to FIG. 1, and

FIG. 3 a representation of a parameter set.

The hearing device system represented in FIG. 1 as a block diagram has a hearing device 1, which consists of electro-acoustic converters, namely at least one microphone, preferably two microphones 2a, 2b as shown, as well as a receiver 3 (loudspeaker). The microphones 2a, 2b are connected to the input of a tranmiassion unit 5 adjustable in its transmission characteristics over an analog to digital converter 4, the transmission unit 5 being connected at its output end to the receiver 3 over a digital to analog converter 6. In the transmission unit 5 a number of different sets of parameters are saved, of which each is assigned to a certain acoustic surrounding situation and determines a transmission characteristic of the transmission unit 5. Such a parameter set 7 is schematically represented in FIG. 3. Furthermore, a signal processing unit a belongs to the hearing device 1, which signal processing unit B consists of a signal analyzing unit 9 and a signal identification unit 10 connected with the signal analyzing unit 9. The signal analyzing unit 9 is connected to the output of the analog to digital converter 4 and is fed at its input end by the input signal of the transmission unit 5. The output of the signal identification unit 10 is connected to the transmission unit 5, to which, furthermore, an input unit 11 is operationally connected. This input unit 11 consists of a remote control unit 12 separated from the hearing device for the shown implemented example, which remote control unit 12 is wirelessly connected to the hearing device 1. This remote control unit 12, only schematically represented in FIG. 2, has a housing 13 in which the necessary electronic components, not further represented, are incorporated as well as two buttons 14 and 15, which are easily identifiable by the hearing device user, according to the shown embodiment by “+” (meaning e.g. “better understanding) and “−” (avaning e.g. “more pleasant hearing”).

It is also possible to integrate the input unit 11—instead of having a remnote control unit 12—into the hearing device 1 and to provide two buttons at the hearing device.

The input unit 11 can also be designed that the input is generated by the hearlng device user through speech control.

The identification of the momentary acoustic surround situation and the automatic adjustment of a hearing program or set of pararmters, respectively, belonging to the identified surround situation, can basically be done as has been already described in WO-A-01/20965 and US-A-2002/0037087. Therefore, reference is made to these printings for a detailed description of the automtic adjustment of the suitable hearing program, which printings are herewith incorporated by reference into this description. As a summary, the automatic hearing program adjustment is performed as followst In the signal analysis unit 9 characteristic, auditory-based features and, if need be, also technically-based features are extracted from the output of the analog to digital converter 4. Based on the features extracted in the signal analysis Unit 9, the momentary acoustic surround situation is determined by recognition of the same or a similar pattern of the extracted features, and a corresponding signal is generated, This output signal, which contains information of the acoustic surround situation, is fed to the transmission unit 5 in which the set of parameters is adjusted determining the mntary transruission characteristic of the transmission Unit 5, which set of parameters is related to the identified acoustic surround situation.

The hearing device user has now the possibility to have influence on the automatically selected set of parameters as described above, i.e. on the adjusted hearing program. Thereto, the input unit ii is used which, as already described, is operationally connected to the transmission unit S. liow this exertion of influence is taking place will be further described in the following by referring to FIG. 3.

It is assumed that the set of parameters 7 adjus ted in the hearing device is shown in FIG. 3 and that this set of parameters 7 consists of the parameters P1-Pn. The nominal value adjusted for each parameter and saved in the transmission unit 5 is represented by a black beam and designated by N. By pushing the buttons 14, 15 the parameters P1-Pn can be changed starting from this nominal value N going into two opposing directions C, D between two end values, which are referenced by A and B in FIG. 3 and which fix a control range. Thereby, not every one of the parameters P1-Pn can be adjusted, which would be very confusing and which would also not be handy. On the contrary, the change of the parameters P1-Pn is performed by pushing the buttons 14, 15 of the remote control =nit 12 for all parameters at the same time and mutually according to predefined rules or set of rules, respectively, saved in the hearing device 1. For different acoustic surround situations, different rules or set of rules, respectively, can be applied. Thereby, every parameter of the adjusted set of parameters can be changed by a different measure, i.e. some of the parameters incur a strong change, whereas other paralmeters are only changed slightly or not at all. This will be further explained by referring to FIG. 3.

In a first case, one considers that the hearing device user has adjusted the parameters in direction of the arrow C by pressing the button 15 (adjusting in the sense of “more pleasant hearing”). The parameters are now adjusted to the value indicated by “X”. In another case, the hearing device user has made a change of the parameters in direction of the arrow D by pressing the button 14 (adjusting in the sense of “better understanding”) so that the parameters are now adjusted to the value indicated by Y. Thereby, the parameter P3 has not been changed in the represented example.

The adjustment of the parameters can be done stepwise or continuous as well as soft, i.e. not hearable, or hard, i.e. hearable.

Thanks to the method according to the present invention and by the hearing device system 1 according to the present invention, the hearing device user is capable to introduce his personal hearing desire, while at the same time an automatic selection of the hearing program best suitable to the momentary acoustic situation is taking place. This will be further clarified by an example.

The hearing device user is in a restaurant. In the background, msic can be heard. At the same table, other people are sitting and are having a discussion. The hearing device is automatically adjusting to this given momentary acoustic surround situation in the manner described above. The hearing device user can now have the desire to better follow up the discussions at the table, or he may want to. hear the music better. In case he wants to follow the discussion better, he pushes the button 14 and adjusts the. parameters P1-Pn in direction of the arrow D in the sense of “better understanding, selective hearing or more intelligibility”. In case the hearing device user wants to listen to the music, he pushes the other button 15 and adjusts the parameters P1-Pn in direction of the arrow C in the sense of “more pleasant hearing or better all-aroud hearing, respectively”.

In the hearing device, a large number of different hearing programs, i.e. the corresponding sets of parameters, can be saved, of which hearing programs the best one is automatically selected according to the momentary acoustic surround situation. The hearing device user can convert his desire in a single manner, namely by pressing a button of a keyboard. Thereby, the hearing desire can change from one acoustic surround situation to the next one, but also within a given acoustic surrounding. The keyboard has only two buttons, but may also have more than two buttons. As a result, the concept according to the present invention nakes it possible to create a hearing device which is easily operable and which is adjusting automatically to a momentary acoustic surround situation and which, in addition, is taking into account the particular hearing desire of a hearing device user.

The described hearing device 1 can be designed in such a way that the corrected sets of parameters, which have been changed by the he,aring device user according to his hearing desire as described above, are saved in the hearing device. In case that the hearing device user changes the assigned parameter set originally saved always and again in the same mamer in a certain acoustic surround situation, i.e. always in the sane direction and to the same extent, this parameter set originally saved is exchanged by the changed corrected parameter set. The corrected parameter set is then used as standard adjustment.

This exchange of the originally saved parameter set by the corrected parameter set can automatically be learnt by the hearing device, e.g. by a neural network, or by a hearing device fitter or by the hearing device user himself.

The hearing device system described above is used in the sense of a hearing aid for the correction of a hearing impairment of a person. The method according to the present invention and the hearing device concept according to the present invention can be used simiarly also for other acoustic convuicatio systemns, as e.g. for radio devices.

1. Method for adapting a hearing device (1) to a momentary acoustic surround situation, comrrising the steps of identifying a momentary acoustic surround situation in the hearing device (1), adjusting a parameter set (7) saved in the hearing device (1) and corresponding to the identified surround situation and adjusting parameters (P1-Pn) of the adjusted parameter set (7) with the aid of an input unit (11) operated by a hearing device user in accordance with the hearing desire of the hearing device user.

2. Method according to claim 1, wherein the parameters (P1-Pn) of the adjusted parameter set (7) are being adjusted starting from an adjusted nominal value (N) within s a preset control range (A-B) in one of two opposing directions (C, D).

3. Method according to claim 1 or 2, wherein the parameters (P1-Pn) of the adjusted parameter set (7) are being adjusted at the same time and according to preset rules or rule sets, respectively.

4. Method according to claim 1, wherein adjusting of the parameters (P1-Pn) of the adjusted parameter set (7) are being performed in the sense of “better selective hearing”, at the one hand, and in the senrse of “better all-around hearing”, at the other hand.

5. Method according to claim 1, further comprising the step of saving parameters adjusted in accordance to the hearing desire of the hearing device user.

6. Method according to claim 5, further comprising the step of exchanging an originally saved parameter set (7) assigned to a specific acoustic surround situation by a corresponding corrected parameter set adjusted by the hearing device user if, for the specific acoustic surround situation, the originally saved parameter set (7) has been changed several times by the hearing device user in the same manner.

7. Method according to claim 1, wherein the step of identifying the momentary acoustic sound situation comprises extracting in the hearing device characteristic features from signals which are recorded from the momentary acoustic surround situation, and identifying the momentary acoustic surround situation based on the extracted features.

8. Hearing device system with a hearing device (1) comprising a transmission unit (5) with an input operationally connected to at least one microphone (2a, 2b) and an output operationally connected to a receiver (6), the transmission unit is adjustable to different momentary transmission characteristics determined by saved parameter sets (7) to adjust its transmission characteristics;
a signal processing unit (8) to which the input signal of the transmission unit (5) is being fed, the signal processing unit (8) identifies a momentary acoustic surround situation and which results in an adjustment of a parameter set (7) belen corresponding to the identified surround situation in the transmission unit (5); and
an input unit (11) operable by the hearing device user and operationally connected to the transmission unit (5), wherein parameters (P1-Pn) of the adjusted parameter set (7) resulting from the momentary transmission characteristics of the transmission unit (5) are further adjusted according to the hearing desire of the hearing device user with the aid of the input unit (11).

9. Hearing device system according to claim 8, wherein the parameters (P1-Pn) of the adjusted parameter set (7) are adjusted starting from an adjusted nominal value (N) within a preset control range (A-B) in one of two opposing directions (C, D) with the aid of the input unit (11), the adjustment of the parameters (Pl-Pn) of the adjisted parameter set (7) being adjusted at the same time and according to preset rules or rule sets, respectively, saved in the hearing device (1).

10. Hearing device system according to claim 9, wherein the adjustment of the parameters of the adjusted parameter set (7) is done with the aid of the input unit (11) in the sense of “better selective hearing”, at one hand, and in the sense of “better all-around hearing”, on the other hand.

11. Hearing device system according to one of the claims 8 to 10, wherein the signal processing unit (8) comprises of:
a signal analyzing unit (9) operationally connected to the at least one microphone (2a, 2b), the signal analyzing unit (9) extracts characteristic features from signals recorded by the at least one microphone (2a, 2b) from the momentary acoustic surround situation; and
a signal identification unit (10) with an input connected to the output of the signal analyzing unit (9) and an output connected to the transmission unit (5), the signal identification unit .(10) identifies the momentary acoustic surround situation based on the extracted characteristic features.

12. Hearing device system according to claim 8, wherein the input unit (11) is designed to manually input-data by a key board (14, 15).

13. Hearing device system according to claim 8, wherein the input unit (11) is designed for speech controlled input of data.

14. Hearing device system according to claim 8, wherein the input unit (11) is designed as remote control (12) separated from the hearing device and is connected to the transmission unit (5) wirelessly.

15. Hearing device system according to claim 8. wherein the input unit (11) is integrated into the hearing device (1).

16. Hearing device system according to claim 12, wherein the key board of the input unit (11) consists of two buttons (14, 15) of which each is used to adjust the parameters (P1-Pn) of the adjusted parameter set (7) in one of the two opposing directions (C, D).
